SEQUENCE LISTING (2 sequences)

SEQ ID NO: 1 - protein, 12 aa, Artificial Sequence
  SYNTHESIZED
PDRKAAVSHWQQ

SEQ ID NO: 2 - protein, 10 aa, Artificial Sequence
  SYNTHESIZED
EQKLISEEDL

(19) United States
(12) Patent Application Publication
(10) Pub. No.: US 2018/0224459 A1
(43) Pub. Date: Aug. 9, 2018

(54) NANOBODY CONJUGATES AND PROTEIN FUSIONS AS BIOANALYTICAL REAGENTS

(71) Applicant: Colorado State University Research Foundation, Fort Collins, CO (US)

(72) Inventors: Brian R. McNaughton, Fort Collins, CO (US); Virginia J. Bruce, Fort Collins, CO (US)

(73) Assignee: Colorado State University Research Foundation, Fort Collins, CO (US)

(21) Appl. No.: 15/891,981

(22) Filed: Feb. 8, 2018

Related U.S. Application Data
(60) Provisional application No. 62/456,519, filed on Feb. 8, 2017.
(60) Provisional application No. 62/471,546, filed on Mar. 15, 2017.

(51) Int. Cl. G01N 33/58; C07K 7/08; C12N 9/02; C12N 9/38

(52) U.S. Cl. CPC G01N 33/581; G01N 33/582; C12Y 113/12; C12N 9/0069; C12N 9/2471; C07K 7/08

(57) ABSTRACT

Systems and methods for detecting a target protein using a nanobody-peptide receptor pair are described.

### CROSS REFERENCE TO RELATED APPLICATIONS

This application claims the benefit of U.S. Provisional Application No. 62/456,519, filed Feb. 8, 2017, and U.S. Provisional Application No. 62/471,546, filed Mar. 15, 2017, the disclosures of which are hereby incorporated by reference in their entirety.

### GOVERNMENTAL RIGHTS

This invention was made with government support under R01GM107520 awarded by the National Institutes of Health. The government has certain rights in the invention.

### FIELD OF THE INVENTION

The present disclosure relates to systems and methods for detecting a target protein using a nanobody-peptide receptor pair.

### SEQUENCE LISTING

The present application contains a Sequence Listing, which has been submitted to the United States Receiving Office (RO/US) via EFS-Web and is hereby incorporated by reference in its entirety.

### BACKGROUND OF THE INVENTION

Enzyme-linked immunosorbent assay (ELISA), flow cytometry, and Western blot are common bioanalytical techniques. Successful execution traditionally requires the use of one or more commercially available antibody-small-molecule dye, or antibody-reporter protein conjugates that recognize relatively short peptide tags (<15 amino acids). However, the size of antibodies and their molecular complexity (by virtue of post-translational disulfide formation and glycosylation) typically requires either expression in mammalian cells or purification from immunized mammals. The preparation and purification of chemical dye- or reporter protein-antibody conjugates is often complicated and expensive, and not commonplace in academic laboratories.

As such, there is a need for simpler protein scaffolds for macromolecular recognition, which can be expressed with relative ease and can be evolved to bind virtually any target.

### SUMMARY OF THE INVENTION

The present disclosure provides a nanobody nanobody-peptide receptor system. The nanobody-peptide tag receptor system comprises a tagged target and a nanobody linked to a reporter, wherein the nanobody has binding affinity to the tag on the tagged target. The target may be selected from a protein, protein fragment, peptide, amino acid, and cell. The tag may be a peptide having 6 to 20 amino acids. In some aspects the tag may be a peptide having the amino acid sequence PDRKAAVSHWQQ (SEQ ID NO. 1). In other aspects, the tag may be a peptide having at least 80% identity to the amino acid sequence PDRKAAVSHWQQ (SEQ ID NO. 1). The reporter may be selected from a reporter protein, dye, and radioisotope. The reporter protein may be selected from fluorescent protein, luciferase, alkaline phosphatase, β-galactosidase, β-lactamase, dihydrofolate reductase, and ubiquitin. In some aspects, the reporter protein may be a luciferase. In further aspects, the luciferase is nLuc.

In another aspect, the nanobody-peptide tag receptor system may be used in an immunological method selected from immunoassays, indirect immunofluorescence, direct immunofluorescence, enzyme-linked immunosorbent assay (ELISA), flow cytometry, fluorescence activated cell sorting (FACS), Western blot, paper-based diagnostics, and microfluidic diagnostics.

In an additional aspect, the present disclosure provides a method of detecting a tagged target. The method may include: obtaining a nanobody having binding affinity for a tag, wherein the nanobody is linked to a reporter; contacting the tagged target with the nanobody, wherein the tag is present on the target; and detecting the reporter. The target may be selected from a protein, protein fragment, peptide, amino acid, and cell. The tag may be a peptide having 6 to 20 amino acids. In some aspects the tag may be a peptide having the amino acid sequence PDRKAAVSHWQQ (SEQ ID NO. 1). In other aspects, the tag may be a peptide having at least 80% identity to the amino acid sequence PDRKAAVSHWQQ (SEQ ID NO. 1). The reporter may be selected from a reporter protein, dye, and radioisotope. The reporter protein may be selected from fluorescent protein, luciferase, alkaline phosphatase, β-galactosidase, β-lactamase, dihydrofolate reductase, and ubiquitin. In some aspects, the reporter protein may be a luciferase. In further aspects, the luciferase is nLuc.

In another aspect, the present disclosure provides a method of detecting a cell. The method may include: obtaining a cell that has been modified to display a tagged protein; contacting the tagged protein with a nanobody linked to a reporter, wherein the nanobody binds the tag on the tagged protein; and detecting the reporter. The tag may be a peptide having 6 to 20 amino acids. In some aspects the tag may be a peptide having the amino acid sequence PDRKAAVSHWQQ (SEQ ID NO. 1). In other aspects, the tag may be a peptide having at least 80% identity to the amino acid sequence PDRKAAVSHWQQ (SEQ ID NO. 1). The reporter may be selected from a reporter protein, dye, and radioisotope. The reporter protein may be selected from fluorescent protein, luciferase, alkaline phosphatase, β-galactosidase, β-lactamase, dihydrofolate reductase, and ubiquitin. In some aspects, the reporter protein may be a luciferase. In further aspects, the luciferase is nLuc.

### BRIEF DESCRIPTION OF THE FIGURES

The application file contains at least one drawing executed in color. Copies of this patent application publication with color drawing(s) will be provided by the Office upon request and payment of the necessary fee.

FIG. 1(A) depicts the architecture of a heavy chain IgG (hcIgG), consisting of two heavy chains (CH3, CH2, VH) connected by disulfide bonds in the hinge region. The “nanobody” subunit is circled. FIG. 1(B) depicts the structure of the recently reported nanobody BC2, bound to its peptide tag (BC2T, PDB: 5IVN).

FIG. 2(A) depicts an Enzyme-Linked Immunosorbent Assay (ELISA). FIG. 2(B) depicts ELISA data. Immobilized GFP was treated with buffer (NT), and either HRX-BCT2, GFPnb-His6, GFPnb-myc, or GFPnb-BC2T, then either anti-His6-HRP, anti-myc-HRP, or the BC2nb-HRP conjugate, and HRP substrate. FIG. 2(C) depicts ELISA data. GFP was immobilized onto streptavidin coated plates, then treated with buffer (NT), HRX-BC2T (HXX-BC2T), GFP nanobody (GFPnb), GFPnb-BC2T (GFPnb-BC2T), or a 1:1 mixture of GFP nanobody and GFPnb-BC2T (1:1 mixture), followed by nLuc substrate. All experiments were performed in triplicate. Error bars represent standard deviation of three experiments. α=anti; NT=no treatment.

FIG. 3 depicts ELISA data using BC2 nanobody-HRP for reading. All lanes did not have anything immobilized on the plate's surface. To test non-specific binding of anti-His6 antibody-HRP, anti-myc antibody-HRP, and BC2nb-HRP, wells were then incubated with just buffer (NT), a protein that does not have affinity for GFP (HRX-BC2T), a protein that does have affinity for GFP but different tags depending on which antibody was used (anti-His6, colored black, anti-myc, colored grey, and BC2 nanobody-HRP, colored white). After a 30 minute incubation with TMB-one substrate plate was read at 655nm. All experiments were performed in triplicate. Error bars represent standard deviation of three experiments. NT=no treatment.

FIG. 4 depicts ELISA data using BC2 nanobody-nLuc for reading. Lanes 1-4 did not have anything immobilized on the plate's surface. To test non-specific binding of BC2nb-nLuc, wells were then incubated with just buffer (NT; lane 1, colored black), a protein that does not have affinity for GFP (HRX-BC2T; lane 2, colored red), a protein that does have affinity for GFP but no BC2T epitope (GFPnb-His6; lane 3, colored orange), and GFP displaying BC2T (lane 4, colored green). Luminescence was read after a 10 minute incubation with NanoGlo substrate. All experiments were performed in triplicate. Error bars represent standard deviation of three experiments. NT=no treatment.

FIG. 5(A) depicts a representation of E. coli engineered for flow cytometry experiments. FIG. 5(B) depicts a Representation of yeast engineered for flow cytometry experiments. FIG. 5(C) shows flow cytometry detection of displayed monomeric streptavidin (mSA2) on the surface of E. coli or yeast, as determined by commercially available antibody a-myc-FITC, or nanobody reagents BC2nb-Cy5, or BC2nb-GFP (for E. coli), or commercially available antibodies a-myc-FITC, or a-HA-FITC, or nanobody reagents BC2nb-Cy5, or BC2nb-GFP (for yeast). All experiments were performed in triplicate. Error bars represent standard deviation of three experiments. α=anti; NT=no treatment.

FIGS. 6(A)-6(B) depict flow cytometry data for display of mSA2 on bacteria after incubation with anti-HA antibody-FITC, anti-myc antibody-FITC, BC2 nanobody—Cy5, or BC2 nanobody-GFP. Non-induced samples are shown as dashed lines and induced samples as solid lines.

FIGS. 7(A)-7(B) depict flow cytometry data for display of mSA2 on yeast after incubation with anti-HA antibody-FITC, anti-myc antibody-FITC, BC2 nanobody—Cy5, or BC2 nanobody-GFP. Non-induced samples are shown as dashed lines and induced samples as solid lines.

FIGS. 8(A)-8(B) depict selectivity for epitope validated via Western blot. FIG. 8(A) shows a 5 μM Coomassie stained gel and Western blot analysis of GFP-BC2T and GFP. Western blot analysis used BC2 nanobody-IRdye800. FIG. 8(B) shows 5 μM Coomassie stained gel and Western blot analysis of GFP-HA and GFP. Western blot analysis used anti-HA antibody and was visualized with Donkey Anti-Rabbit IgG Alexa Fluor 790.

FIGS. 9(A)-9(C) depict Coomassie stained gels and Western blot analysis of GFP-HA, GFP-myc, and GFP-BCT2. FIGS. 9(A)-(C), left gels, are Coomassie stained polyacrylamide gels following loading with 20, 10, 5, or 1 μM GFP-HA, GFP-myc, or GFP-BCT2, and electrophoresis. FIGS. 9(A)-(C), right gels, are Western blot data for the GFP-HA/anti-HA; GFP-myc/anti-myc, or; GFP-BC2T/BC2nb pairs, respectively. α=anti.

### DETAILED DESCRIPTION OF THE INVENTION

While full-length antibodies are used in bioanalytical techniques and sensor platforms, their size and complexity requires isolation from mammalian cells or immunized mammals (principally goat, mouse, or rabbit). This complicated production greatly adds to the cost of antibody-based reagents, which has negative consequences in basic research and commercial diagnostics development and application. Moreover, the inability of most academic labs to express and purify full-length antibodies, and chemically conjugate them to chemical dyes or reporter proteins, makes it challenging to prepare reagents in the academic lab itself.

Described herein are systems and methods for detecting a target protein utilizing a nanobody-peptide receptor pair comprising nanobody linked to a reporter and a peptide tag. It was unexpectedly discovered that the described nanobody-peptide receptor pair systems (also referred to herein as “nanobody systems”) can be used successfully in place of antibody-based reagents in a wide variety of bioanalytical techniques and sensor platforms. These systems can be tailored to the needs of a particular bioassay and the reagents can be developed with less expensive and sophisticated laboratory equipment than traditional antibody-based reagents. The nanobodies in the systems and methods disclosed allow for the use of CDR loops that are capable of binding peptides tags with higher specificity as compared to antibodies. The nanobodies of this disclosure, in some aspects, are capable of being expressed in bacteria such as E. coli, allowing for good production. Moreover, in some aspects, these systems do not require a difficult chemical conjugation—a process that most academic labs are not equipped to handle. Accordingly, the disclosed nanobody systems and methods represent an excellent tool for protein detection. Various aspects of the nanobody systems and methods of use thereof are described in detail below.

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms as used herein and in the claims shall include pluralities and plural terms shall include the singular. The use of “or” means “and/or” unless stated otherwise. Furthermore, the use of the term “including,” as well as other forms, such as “includes” and “included,” is not limiting.

As will be realized, the disclosed aspects are capable of modifications in various aspects, all without departing from the spirit and scope of the present disclosure. Accordingly, all sections of the present disclosure, including the Summary, Drawings, and Detailed Description are to be regarded as illustrative in nature and not restrictive.

### Nanobody-Peptide Tag Receptor Pairs

The nanobody systems of the present disclosure comprise a nanobody linked to a reporter (also referred to as a “nanobody reporter”) that recognizes a target protein, protein fragment, peptide, or amino acid (also referred to as a “peptide tag” or “tag”). The nanobody systems of the present disclosure may also comprise the tag that the nanobody reporter recognizes.

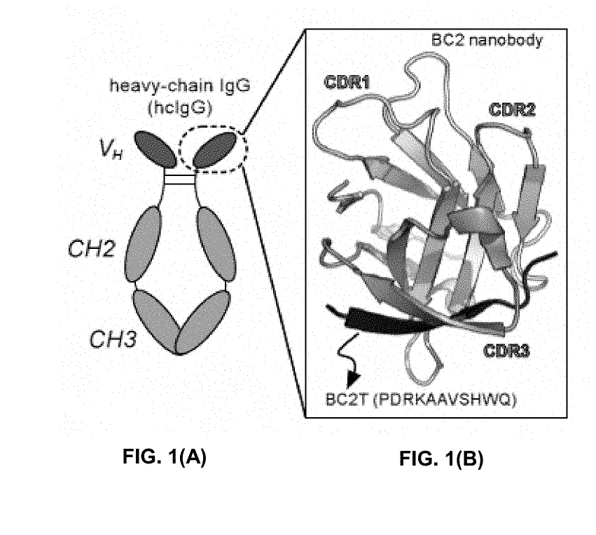

As used herein, a “nanobody” refers to a single-domain antibody, generally designated sdAb, which is an antibody fragment consisting of a single monomeric variable antibody domain which is able to bind selectively to an antigen. A nanobody may comprise heavy chain variable domains or light chain variable domains. Specifically, a nanobody of the disclosure comprises heavy chain variable domain. By way of a non-limiting example, FIG. 1(B) depicts an isolated VH domain of heavy-chain IgGs (a nanobody). A nanobody may be derived from camelids (V_{H}H fragments) or cartilaginous fishes (V_{NAR} fragments). Alternatively, a nanobody may be derived from splitting the dimeric variable domains from IgG into monomers.

A nanobody comprises a variable region primarily responsible for antigen recognition and binding and a framework region (FIG. 1(B)). The “variable region,” also called the “complementarity determining region” (CDR), comprises loops which differ extensively in size and sequence based on antigen recognition. CDRs are generally responsible for the binding specificity of the nanobody. Distinct from the CDRs is the framework region. The framework region is relatively conserved and assists in overall protein structure.

Nanobodies of the present disclosure can recognize a diverse array of target proteins, proteins fragments, peptides, or amino acids (tags), through interactions involving one or more CDR loops (CDR 1-3, FIG. 1(B)). The nanobody may naturally specifically bind a tag or the nanobody may be modified to specifically bind a tag. A nanobody that naturally specifically binds a tag may be obtained by immunizing a subject capable of producing a nanobody with a tag and isolating a nanobody from the serum of the subject. Alternatively, CDRs known to specifically bind a target protein may be grafted onto a nanobody framework region. The assignment of amino acid sequences to each CDR may be in accordance with known conventions (See, Kabat “Sequences of Proteins of Immunological Interest” National Institutes of Health, Bethesda, Md., 1987 and 1991; Chothia, et al, J. Mol. Bio. (1987) 196:901-917; Chothia, et al., Nature (1989) 342:878-883, the disclosures of which are incorporated by reference in their entirety). Further, high-throughput screening may be used to identify a nanobody that specifically binds to a tag. Still further, in vitro evolution methods may be used to generate a nanobody that specifically binds a tag. The phrase “in vitro evolution” generally means any method of selecting for a nanobody that binds to a target protein. In vitro evolution is also known as “in vitro selection”, “SELEX,” or “systematic evolution of ligands by exponential enrichment.” Briefly, in vitro evolution involves screening a pool of random nanobodies for a particular nanobody that binds to a tag or has a particular activity that is selectable. Accordingly, in vitro evolution is used to generate nanobodies that specifically bind to distinct epitopes of any given tag.

The tags may be 6 to 20 amino acids in length. In some aspects, the tags are at least 6 amino acids in length, at least 7 amino acids in length, at least 8 amino acids in length, at least 9 amino acids in length, at least 10 amino acids in length, at least 11 amino acids in length, at least 12 amino acids in length, at least 13 amino acids in length, at least 14 amino acids in length, at least 15 amino acids in length, at least 16 amino acids in length, at least 17 amino acids in length, at least 18 amino acids in length, at least 19 amino acids in length, or at least 20 amino acids in length. In further aspects the tags are 12 amino acids in length.

Non-limiting examples of tags include: BC2 tag, FLAG7, polyhistidine tag (his6), myelocytomatosis viral oncogene8 (myc), synthetic streptavidin binding Strep-tag, and influenza hemmaglutinin (HA).

In some aspects, the nanobody reporter binds to a tag that is present on a target. By way of non-limiting examples, the target can be a protein, protein fragment, polypeptide, amino acid, or cell. In some aspects, the tag can be naturally occurring on the target. In other aspects, the target can be engineered to contain or express the tag.

The tag may be present on a protein, protein fragment, polypeptide, or amino acid. The protein, protein fragment, polypeptide, or amino acid target may be engineered to express the tag using appropriate techniques well known to those of skill in the art.

The tag may be present on a cell. The tag may be intracellular or extracellular to a cell. A cell may be engineering to express the tag. In some aspects, a cell may be engineered to express the tag on the cell surface. In other aspects, a cell may be engineered to express the tag inside the cell. The tag may be attached to or be part of another protein, protein fragment, polypeptide, or amino acid that expressed by or attached to a cell.

The tag expressing cell may be any type of cell. In some aspects the cell may a bacteria, yeast, or animal cell. The cell expressing the tag may be in vitro, such as a commercially available cell line (e.g. American Type Culture Collection (ATCC)). Alternatively, a tag expressing cell may be in vivo; i.e., the cell may be disposed in a subject. A subject may be a human or a non-human animal. Non-limiting examples of non-human animals include companion animals (e.g., cats, dogs, horses, rabbits, gerbils), agricultural animals (e.g., cows, pigs, sheep, goats, fowl), research animals (e.g., rats, mice, rabbits, primates), and zoo animals (e.g., lions, tiger, elephants, and the like).

In one aspect, the tag in the nanobody system is BC2T. BC2T is a short peptide having the amino acid sequence PDRKAAVSHWQQ (SEQ ID NO. 1). In some aspects the tag comprises at least 80% identity to PDRKAAVSHWQQ (SEQ ID NO. 1). For example, the tag may have about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to PDRKAAVSHWQQ (SEQ ID NO. 1).

A nanobody referred to as BC2 binds the BC2T with excellent affinity (KD ˜1.4 nM) and selectivity, principally through interactions involving CDR 3 (FIG. 1(B)). In one aspect, the nanobody reporter of the present disclosure is BC2 linked to a reporter.

The nanobodies of the present disclosure are linked to reporters. In some aspects, the reporters may be reporter proteins, dyes, or radioisotopes.

“Reporter protein,” as used herein, refers to any protein capable of generating a detectable signal. Reporter proteins typically fluoresce, or catalyze a colorimetric or fluorescent reaction. Any suitable reporter protein, as understood by one of skill in the art, could be used. In some aspects, the reporter protein may be selected from fluorescent protein, luciferase, alkaline phosphatase, β-galactosidase, β-lactamase, dihydrofolate reductase, ubiquitin, and variants thereof.

Non-limiting examples of reporter proteins that fluoresce include green fluorescent proteins (GFP), red fluorescent proteins (YFP), yellow fluorescent proteins (YFP), blue fluorescent proteins such as TagBFP (Evrogen), cyan fluorescent proteins, orange fluorescent proteins, and far-red fluorescent proteins such as mNeptune. Non-limiting examples of green fluorescent proteins include: mTagBFP2 (Evrogen), EGFP, Emerald, Superfolder GFP, Monomeric Azami Green (MBL International), TagGFP2 (Evrogen), mUKG, mWasabi (Allele Biotech), Clover, and mNeonGreen (Allele Biotech). Non-limiting examples of red fluorescent proteins include: mRaspberry, mCherry, mStrawberry, mTangerine, tdTomato, TagRFP (Evrogen), TagRFP-T, maple, mRuby, and mRuby2. Non-limiting examples of cyan fluorescent proteins include: monomeric Midoriishi-Cyan (MBL International); Tag CFP (Evrogen); and mTFP1 (Allele Biotech). Non-limiting examples of yellow fluorescent proteins include: EYFP, Citrine, Venus, SYFP2, and TagYFP (Evrogen). The sequences of fluorescent proteins and their characteristics (e.g., excitation and emission wavelengths, extinction coefficients, brightness, and pKa) are generally detailed in the source literature well known to those of routine skill in the art.

Non-limiting examples of reporter proteins that catalyze a colorimetric or fluorescent reaction include luciferases, alkaline phosphatase, β-galactosidase, β-lactamase, and dihydrofolate reductase. Any suitable luciferase, as understood by one of skill in the art, could be used. Some non-limiting examples of luciferase include, but are not limited to, nanoluciferase (e.g. NanoLuc® Promega), firefly luciferase, Metridia luciferase, and dinoflagellate luciferase. The sequences of luciferase proteins and their characteristics are generally detailed in the source literature well known to those of routine skill in the art.

“Dye,” as used herein, refers to a chemical compound or polypeptide that is capable of generating a detectable signal. As used herein, the term “dye” refers to both single and tandem dyes. Any suitable dye, as understood by one of skill in the art, could be used. Non-limiting examples of a dye includes fluorescent chemical dyes that can re-emit light upon excitation. Non-limiting examples of fluorescent chemical dyes include phycoerythin (PE), cyanine, Brilliant Violet™ (BD Horizon) (e.g., BV421, BV510, BV605, BV650, BV711, BV786), Brilliant Ultraviolet™ (BD Horizon) (BUV496), fluorescein (fluorescein isotheiocyanate (FITC), rhodamine (tetramethyl rhodamine isothocyanate, TRICTC), allophycocyanin (APC), phycoerythrin and cyanine dye (PE-Cy), peridinin chlorophyll (PerCP), propidium iodide (PI), allophycocyanine (APC), eFluor, Alexa Fluor, and AmCyan.

“Radioisotope,” as used herein, refers to a radioactive isotope. Any suitable radioisotope, as understood by those of skill in the art can be used and conjugated using methods known to those of skill. Non-limiting examples of radioactive isotopes include, but are not limited to, ¹²⁵I, ¹³¹I, ¹²³I, ¹¹¹In, ³H, ¹⁴C, ^{99m}Tc, ³⁵S, ²¹¹At, ⁹⁰Y, ¹⁸⁶Re, ¹⁸⁸Re, ¹⁵³Sm, ²¹²Bi, and ³²P.

In one aspect, the nanobodies of the present disclosure are conjugated to dyes or radioisotopes (referred to herein as “nanobody conjugate”). A “nanbody conjugate,” as used herein, refers to a nanobody that is operably linked to a dye or radioisotopes. The nanobody can be linked (“conjugated”) to the dye or radioisotopes by appropriate methods known to those of skill in the art.

In another aspect, the nanobodies of the present disclosure are fused to reporter proteins (referred to as “nanobody fusion”). A “nanobody fusion,” as used herein, refers to a nanobody that is operably linked to a reporter protein. The nanobody can be linked (“fused”) to the reporter protein by appropriate methods known to those of skill in the art.

Fusion of the reporter protein to the nanobody has unexpected advantages as compared to the conjugation of dyes. Conjugation can be expensive and difficult for most laboratories. It was unexpectedly discovered that the nanobody fusions of this disclosure express well in bacteria, such as E. coli, and can be easily purified. It was also unexpectedly discovered that nanobody fusions of this disclosure can successfully be used in a wide range of bioanalytical techniques including immunological methods that use specific antigen-antibody recognition. As such, the nanobody fusions of this disclosure provide methods and systems that can be readily utilized by laboratories.

The reporter may be linked to the nanobody or indirectly to the nanobody via a linker. It is to be understood that linking the nanobody to the reporter, or fusion of the nanobody to the linker and connection of the linker to the reporter, will not adversely affect either the binding function of the nanobody or the function of the reporter. Suitable linkers include amino acid chains and alkyl chains functionalized with reactive groups for coupling to both the nanobody and the reporter. An amino acid chain linker may be about 1 to about 40 residues, more often about 1 to about 10 residues. Typical amino acids residues used for linking are tyrosine, cysteine, lysine, glutamic and aspartic acid, and the like.

### Nanobody Construct

In an aspect, the present disclosure provides a nanobody construct. A nanobody construct of the disclosure is a polynucleotide sequence encoding a polypeptide, the polypeptide comprising a nanobody. Further, in some aspects, a nanobody construct of the disclosure is a polynucleotide sequence encoding a polypeptide, the polypeptide comprising a cell-penetrating nanobody fused to a reporter protein. As used herein, the terms “polynucleotide sequence of the disclosure” and “nanobody construct” are interchangeable. The present disclosure also provides isolated polypeptides encoded by nanobody constructs, vectors comprising nanobody constructs, and isolated cells comprising said vectors. The present disclosure further provides isolated polypeptides encoded by nanobody constructs, vectors comprising nanobody constructs, and isolated cells comprising said vectors.

Polynucleotide Sequence

A nanobody construct of the disclosure is a polynucleotide sequence encoding a polypeptide, the polypeptide comprising a nanobody. The polypeptide comprising the cell-penetrating nanobody may further comprise a reporter protein. Additionally, the polynucleotide sequence of the disclosure may encode a polypeptide comprising the nanobody that further comprises a linker linking the nanobody to the reporter protein. The polynucleotide sequence of the disclosure may encode a polypeptide comprising the nanobody that further comprises a linker that allows for linking the nanobody to a dye or radioisotope. The nanobody is capable of specifically binding to tag.

Each of the above embodiments may optionally comprise a signal peptide and/or a purification moiety. When present, typically the polynucleotide sequence encoding the signal peptide is at the N-terminus of the nanobody construct and the polynucleotide sequence encoding the purification moiety is at the C-terminus of the nanobody construct. Alternatively, the polynucleotide sequence encoding the signal peptide and the polynucleotide sequence encoding the purification moiety are both at the N-terminus of the nanobody construct. The choice of polynucleotide sequence encoding the signal peptide can and will vary depending on a variety factors including, but not limited to, the desired cellular location and type of cell. Suitable polynucleotide sequence encoding signal peptides are known in the art, as are polypeptide sequences encoded therefrom. Similarly, the choice of purification moiety can and will vary. Suitable purification moieties are known in the art, as are the polynucleotide sequences encoding them. In a specific embodiment, the purification moiety is a histidine tag.

Polynucleotide sequences of the disclosure may be produced from nucleic acids molecules using molecular biological methods known to in the art. Any of the methods known to one skilled in the art for the amplification of polynucleotide fragments and insertion of polynucleotide fragments into a vector may be used to construct the polynucleotide sequences of the disclosure. These methods may include in vitro recombinant DNA and synthetic techniques and in vivo recombinations (See Sambrook et al. Molecular Cloning, A Laboratory Manual, Cold Spring Harbor Laboratory; Current Protocols in Molecular Biology, Eds. Ausubel, et al., Greene Publ. Assoc., Wiley-Interscience, NY, the disclosures of which are hereby incorporated by reference in its entirety).

Polypeptide Sequence

In another aspect, the present disclosure provides one or more isolated polypeptide(s) encoded by a polynucleotide sequence of the disclosure. Polynucleotide sequences of the disclosure are described in detail above, and are hereby incorporated by reference into this section. An isolated polypeptide of the disclosure comprises a nanobody. An isolated polypeptide of the disclosure may further comprise a reporter protein. Additionally, the polypeptide comprising the nanobody may further comprise a linker. The linker may link nanobody to the reporter protein. The nanobody is capable specifically binding to tag.

Isolated polypeptides of the disclosure may be produced from nucleic acids molecules using molecular biological methods known to in the art. Generally speaking, a polynucleotide sequence encoding the polypeptide is inserted into a vector that is able to express the polypeptide when introduced into an appropriate host cell. Appropriate host cells include, but are not limited to, bacterial, yeast, insect, and mammalian cells. Appropriate host cells are known to those of skill in the art. A non-limiting example of an appropriate host cell is E. coli. Once expressed, polypeptides may be obtained from cells using common purification methods. For example, if the polypeptide has a secretion signal, expressed polypeptides may be isolated from cell culture supernatant. Alternatively, polypeptides lacking a secretion signal may be purified from inclusion bodies and/or cell extract. Polypeptides of the disclosure may be isolated from culture supernatant, inclusion bodies or cell extract using any methods known to one of skill in the art, including for example, by chromatography (e.g., ion exchange, affinity, particularly by affinity for the specific antigen after Protein A, and sizing column chromatography), centrifugation, differential solubility, e.g. ammonium sulfate precipitation, or by any other standard technique for the purification of proteins; see, e.g., Scopes, “Protein Purification”, Springer Verlag, N.Y. (1982). Isolation of polypeptides is greatly aided when the polypeptide comprises a purification moiety.

Vector

In another aspect, the present disclosure provides a vector comprising a nanobody construct of the disclosure. As used herein, a vector is defined as a nucleic acid molecule used as a vehicle to transfer genetic material. Vectors include but are not limited to, plasmids, phasmids, cosmids, transposable elements, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus, herpes virus vectors, vaccinia virus vectors, Harvey murine sarcoma virus vectors, murine mammary tumor virus vectors, Rous sarcoma virus vectors.

Specifically, the vector is an expression vector. The vector may have a high copy number, an intermediate copy number, or a low copy number. The copy number may be utilized to control the expression level for the nanobody construct, and as a means to control the expression vector's stability. In one embodiment, a high copy number vector may be utilized. A high copy number vector may have at least 31, 35, 40, 45, 50, 55, 60, 65, 70, 75, 80, 85, 90, 95, or 100 copies per bacterial cell. In other embodiments, the high copy number vector may have at least 100, 125, 150, 175, 200, 225, 250, 275, 300, 325, 350, 375, or 400 copies per host cell. In an alternative embodiment, a low copy number vector may be utilized. For example, a low copy number vector may have one or at least two, three, four, five, six, seven, eight, nine, or ten copies per host cell. In another embodiment, an intermediate copy number vector may be used. For instance, an intermediate copy number vector may have at least 10, 11, 12, 13, 14, 15, 16, 17, 18, 19, 20, 21, 22, 23, 24, 25, 26, 27, 28, 29, or 30 copies per host cell.

Expression vectors typically contain one or more of the following elements: promoters, terminators, ribosomal binding sites, and IRES. The term “promoter,” as used herein, may mean a synthetic or naturally-derived molecule that is capable of conferring, activating, or enhancing expression of a nucleic acid. A promoter may comprise one or more specific transcriptional regulatory sequences to further enhance expression and/or to alter the spatial expression and/or temporal expression of a nucleic acid. A promoter may be constitutive, inducible/repressible or cell type specific. In certain embodiments, the promoter may be constitutive. Non-limiting examples of constitutive promoters include CMV, UBC, EF1α, SV40, PGK, CAG, CBA/CAGGS/ACTB, CBh, MeCP2, U6, and H1. Non-limiting examples of inducible promoters include tetracycline, heat shock, steroid hormone, heavy metal, phorbol ester, adenovirus E1A element, interferon, and serum inducible promoters. Alternatively, the promoter may be cell type specific.

Expression of the nucleic acid molecules may be regulated by a second nucleic acid sequence so that the molecule is expressed in a host transformed with the recombinant DNA molecule. For example, expression of the nucleic acid molecules may be controlled by any promoter/enhancer element known in the art.

A nucleic acid encoding a nanobody construct may also be operably linked to a nucleotide sequence encoding a selectable marker. A selectable marker may be used to efficiently select and identify cells that have integrated the exogenous nucleic acids. Selectable markers give the cell receiving the exogenous nucleic acid a selection advantage, such as resistance towards a certain toxin or antibiotic. Suitable examples of antibiotic resistance markers include, but are not limited to, those coding for proteins that impart resistance to kanamycin, spectomycin, neomycin, gentamycin (G418), ampicillin, tetracycline, chloramphenicol, puromycin, hygromycin, zeocin, and blasticidin.

An expression vector encoding a nanobody construct may be delivered to the cell using a viral vector or via a non-viral method of transfer. Viral vectors suitable for introducing nucleic acids into cells include retroviruses, adenoviruses, adeno-associated viruses, rhabdoviruses, and herpes viruses. Non-viral methods of nucleic acid transfer include naked nucleic acid, liposomes, and protein/nucleic acid conjugates. An expression construct encoding a nanobody construct that is introduced to the cell may be linear or circular, may be single-stranded or double-stranded, and may be DNA, RNA, or any modification or combination thereof.

An expression construct encoding a nanobody construct may be introduced into the cell by transfection. Methods for transfecting nucleic acids are well known to persons skilled in the art. Transfection methods include, but are not limited to, viral transduction, cationic transfection, liposome transfection, dendrimer transfection, electroporation, heat shock, nucleofection transfection, magnetofection, nanoparticles, biolistic particle delivery (gene gun), and proprietary transfection reagents such as Lipofectamine, Dojindo Hilymax, Fugene, jetPEI, Effectene, or DreamFect. Upon introduction into the cell, an expression construct encoding a nanobody construct may be integrated into a chromosome. Integration of the expression construct encoding a nanobody construct into a cellular chromosome may be achieved with a mobile element.

Cells transfected with the expression construct encoding a nanobody construct generally will be grown under selection to isolate and expand cells in which the nucleic acid has integrated into a chromosome. Cells in which the expression construct encoding a nanobody construct has been chromosomally integrated may be maintained by continuous selection with the selectable marker. The presence and maintenance of the integrated exogenous nucleic acid sequence may be verified using standard techniques known to persons skilled in the art such as Southern blots, amplification of specific nucleic acid sequences using the polymerase chain reaction (PCR), and/or nucleotide sequencing.

Nucleic acid molecules are inserted into a vector that is able to express the fusion polypeptides when introduced into an appropriate host cell. Appropriate host cells include, but are not limited to, bacterial, yeast, insect, and mammalian cells.

Isolated Cell

In another aspect, the present disclosure provides an isolated cell comprising a vector of the disclosure. The cell may be a prokaryotic cell or a eukaryotic cell. Appropriate cells include, but are not limited to, bacterial, yeast, insect, and mammalian cells.

The isolated host cell comprising a vector of the disclosure may be used to produce a polypeptide encoded by a nanobody construct of the disclosure. Generally, production of a polypeptide involves transfecting isolated host cells with a vector comprising a nanobody construct and then culturing the cells so that they transcribe and translate the desired polypeptide. The isolated host cells may then be lysed to extract the expressed polypeptide for subsequent purification. “Isolated host cells” are cells which have been removed from an organism and/or are maintained in vitro in substantially pure cultures. A wide variety of cell types can be used as isolated host cells, including both prokaryotic and eukaryotic cells. Isolated cells include, without limitation, bacterial cells, fungal cells, yeast cells, insect cells, and mammalian cells.

In one embodiment, the isolated host cell is characterized in that after transformation with a vector of the disclosure, it produces the desired polypeptide for subsequent purification. Such a system may be used for protein expression and purification as is standard in the art. In some embodiments, the host cell is a prokaryotic cell. Non-limiting examples of suitable prokaryotic cells include E. coli and other Enterobacteriaceae, Escherichia sp., Campylobacter sp., Wolinella sp., Desulfovibrio sp. Vibrio sp., Pseudomonas sp. Bacillus sp., Listeria sp., Staphylococcus sp., Streptococcus sp., Peptostreptococcus sp., Megasphaera sp., Pectinatus sp., Selenomonas sp., Zymophilus sp., Actinomyces sp., Arthrobacter sp., Frankia sp., Micromonospora sp., Nocardia sp., Propionibacterium sp., Streptomyces sp., Lactobacillus sp., Lactococcus sp., Leuconostoc sp., Pediococcus sp., Acetobacterium sp., Eubacterium sp., Heliobacterium sp., Heliospirillum sp., Sporomusa sp., Spiroplasma sp., Ureaplasma sp., Erysipelothrix sp., Corynebacterium sp. Enterococcus sp., Clostridium sp., Mycoplasma sp., Mycobacterium sp., Actinobacteria sp., Salmonella sp., Shigella sp., Moraxella sp., Helicobacter sp, Stenotrophomonas sp., Micrococcus sp., Neisseria sp., Bdellovibrio sp., Hemophilus sp., Klebsiella sp., Proteus mirabilis, Enterobacter cloacae, Serratia sp., Citrobacter sp., Proteus sp., Serratia sp., Yersinia sp., Acinetobacter sp., Actinobacillus sp. Bordetella sp., Brucella sp., Capnocytophaga sp., Cardiobacterium sp., Eikenella sp., Francisella sp., Haemophilus sp., Kingella sp., Pasteurella sp., Flavobacterium sp. Xanthomonas sp., Burkholderia sp., Aeromonas sp., Plesiomonas sp., Legionella sp. and alpha-proteobaeteria such as Wolbachia sp., cyanobacteria, spirochaetes, green sulfur and green non-sulfur bacteria, Gram-negative cocci, Gram negative bacilli which are fastidious, Enterobacteriaceae-glucose-fermenting gram-negative bacilli, Gram negative bacilli-non-glucose fermenters, Gram negative bacilli-glucose fermenting, oxidase positive.

Particularly useful bacterial host cells for protein expression include Gram negative bacteria, such as Escherichia coli, Pseudomonas fluorescens, Pseudomonas haloplanctis, Pseudomonas putida AC10, Pseudomonas pseudoflava, Bartonella henselae, Pseudomonas syringae, Caulobacter crescentus, Zymomonas mobilis, Rhizobium meliloti, Myxococcus xanthus and Gram positive bacteria such as Bacillus subtilis, Corynebacterium, Streptococcus cremoris, Streptococcus lividans, and Streptomyces lividans. E. coli is one of the most widely used expression hosts. Accordingly, the techniques for overexpression in E. coli are well developed and readily available to one of skill in the art. Further, Pseudomonas fluorescens, is commonly used for high level production of recombinant proteins (i.e. for the development bio-therapeutics and vaccines).

### Methods

In one aspect, a nanobody system of the disclosure may be used in a method of detecting a target. The method may comprise: obtaining a nanobody having binding affinity for a tag, wherein the nanobody is linked to a reporter; contacting a target with the nanobody, wherein the tag is present on the target; and detecting the reporter. In some aspects, the target can be a protein, protein fragment, polypeptide, amino acid, or cell. In some aspects, the reporter is a protein reporter, dye, or radioisotope.

In another aspect, the nanobody reporter and tag can be used as reagents in wide range of bioanalytical techniques. In some aspects these bioanalytical techniques include immunological methods that use specific antigen-antibody recognition. Non-limiting examples of immunological methods include immunoassays, indirect immunofluorescence, direct immunofluorescence, enzyme-linked immunosorbent assay (ELISA), flow cytometry, fluorescence activated cell sorting (FACS), Western blot, paper-based diagnostics, and microfluidic-based diagnostics.

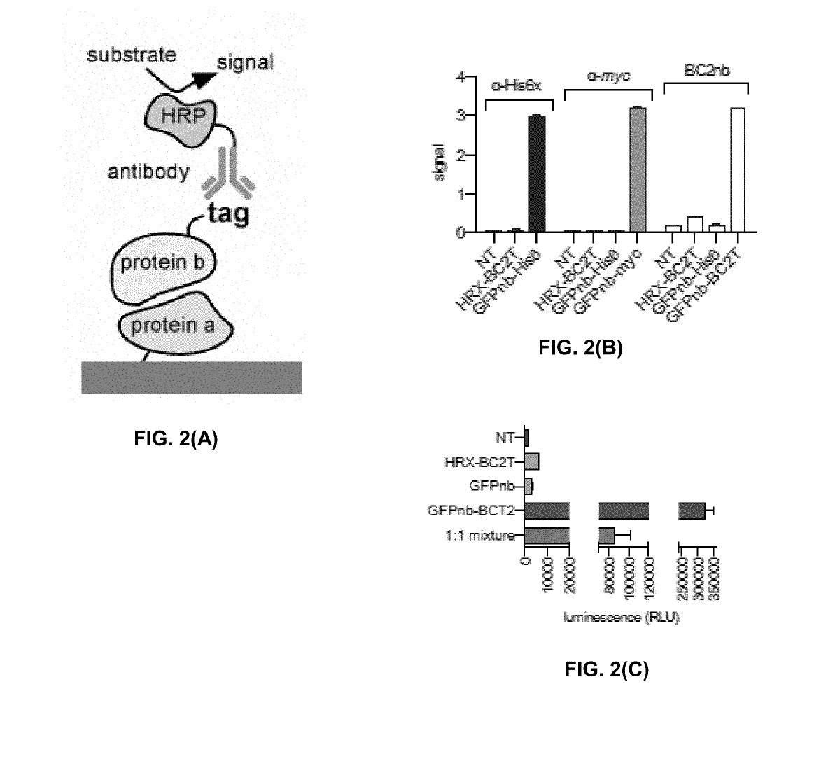

By way of a non-limiting example, the nanobody reporter described herein can be used in ELISA. ELISA typically requires (1) immobilization of a protein (“protein A”) onto a surface (2) incubation with a binding partner (“protein B”) equipped with a small peptide tag; (3) treatment with an antibody-reporter protein conjugate, which recognizes the peptide tag, and generates a signal following addition of a small-molecule substrate (FIG. 2(A)). The presently described nanobody reporter can be used in place of the antibody-reporter protein conjugate in a traditional ELISA. In such an instance, protein B is equipped with a small peptide tag that is recognized by the nanobody reporter. After immobilization of protein A onto a surface and incubation with protein B, treatment with a nanobody reporter, which recognizes the peptide tag occurs and generates a signal following addition of a small-molecule substrate.

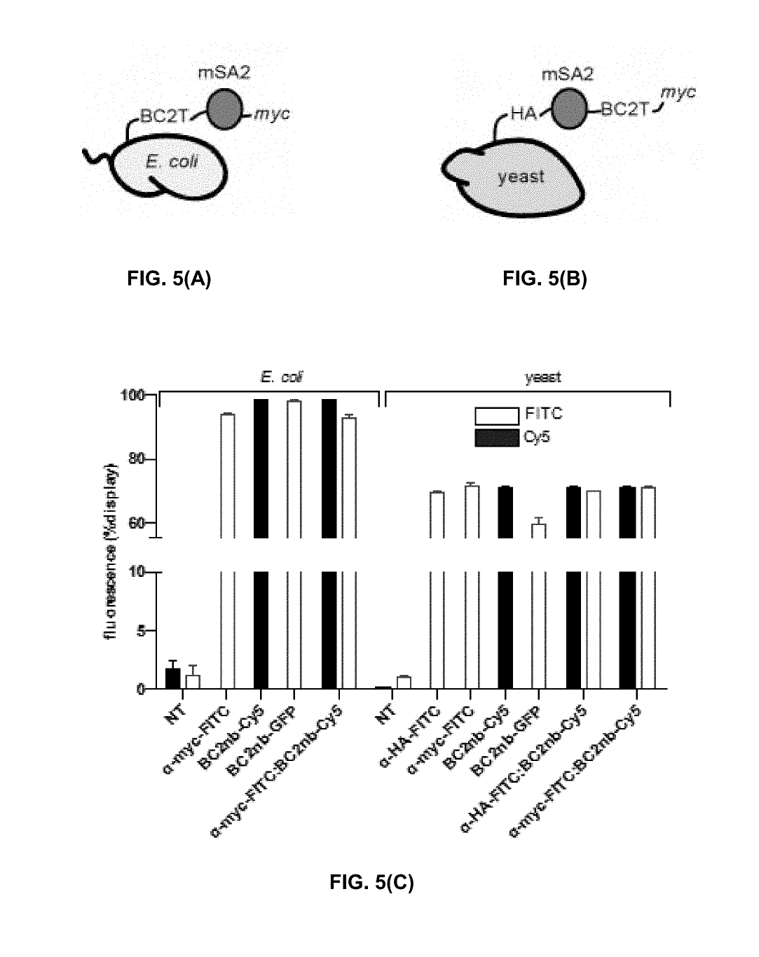

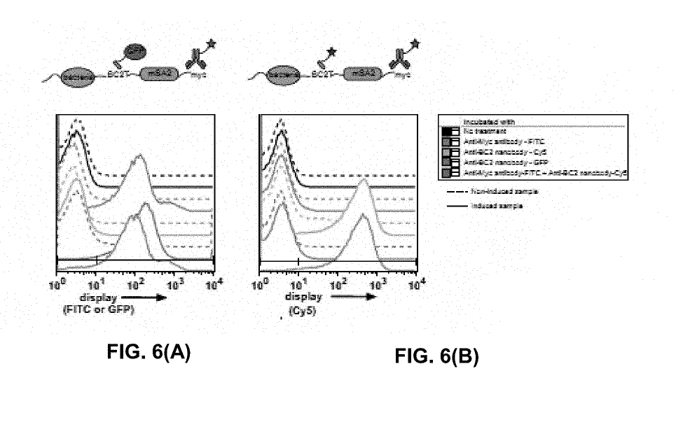

By way of another non-limiting example, the nanobody reporter described herein can be used in flow cytometry. In a typical flow cytometry experiment, bacteria or yeast display a peptide or protein that is flanked by a peptide tag recognized by a commercial antibody-fluorescent dye conjugate. Interaction between the tag and antibody-reporter conjugate allows researchers to quantitate display efficiency. The presently described nanobody reporter can be used in place of the antibody-reporter conjugate. As depicted in FIG. 5(A) and FIG. 6(A), bacteria or yeast may be engineered to display a small protein with a flanking N-terminal and/or C-terminal tag. The bacteria or yeast cells are then treated with a nanobody reporter of the present disclosure, which recognizes the tag. Following washing steps to remove unbound material, the cells are then analyzed by flow cytometry.

By way of another non-limiting example, the nanobody reporter described herein can be used in Western blot. Execution of a Western blot typically requires: (1) denaturation of proteins from cell lysate; (2) separation of proteins based on their size via SDS-PolyAcrylamide Gel Electrophoresis (SDS-PAGE); (3) electrophoretic transfer of separated proteins to a membrane; (4) treatment of the protein-bound membrane with a primary antibody that either recognizes a specific protein, or a specific peptide tag, and; (5) treatment with a secondary antibody-dye conjugate, which serves to illuminate the primary antibody-bound protein. To function in this context, the nanobody reporter must recognize the tag following a chemical denaturation step (and subsequent denaturation of the protein to which it is attached). For this reason, many antibodies are not suitable for Western blot analysis. In some aspects, the presently described nanobody reporters can be used in Western blots wherein the nanobody reporter recognizes a tag on the primary antibody. In other embodiments, the presently described nanobody reporters can be used in Western blots wherein the nanobody reporter recognizes a tag that is displayed on a protein that was separated and transferred to a membrane.

It will be appreciated by those of skill in the art, that the nanobody reporters and systems describes herein can be used as reagents in any appropriate bioanalytical technique and not just the examples described above.

### Kits

The present disclosure also provides a kit for detecting a protein, protein fragment, polypeptide, or amino acid. In some embodiments the kit allows for conducting all or portions of an immunological method, such as those listed above.

A kit may comprise, for example, a nanobody reporter, wherein the nanobody recognizes a target protein (tag) as disclosed herein. The kit may further comprise additional materials or reagents for tagging a protein, protein fragment, polypeptide, or amino acid with the target protein (tag). In some aspects the kit may comprise additional materials or reagents for tagging a cell with the target protein (tag) or expressing the target protein (tag) intracellularly or on the cell's surface. In other aspects, the kit may comprise a protein, protein fragment, peptide, or amino acid that contains the target protein (tag). The kit may further include reagents for carrying out detection of the reporter.

It is contemplated for example that one or more of the presently disclosed nanobody systems can be provided in the form of a kit with one or more containers such as vials or bottles, with each container containing separate reagents and washing reagents employed in an assay. The kit can comprise at least one container for conducting the assay, and/or a buffer, such as an assay buffer or a wash buffer, either one of which can be provided as a concentrated solution, a substrate solution for the reporter protein or dye, or a stop solution. The kit may comprise all components, e.g., reagents, standards, buffers, diluents, etc., which are necessary to perform the assay. The kit may contain instructions for determining the presence or amount of any reporter, in paper form or computer-readable form, such as a disk, CD, DVD, or the like, and/or may be made available online.

Optionally, the kit includes quality control components (for example, sensitivity panels, calibrators, and positive controls). Preparation of quality control reagents is well-known in the art and is described on insert sheets for a variety of immunoassay products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, enzyme substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary. Where appropriate, the kit optionally also can contain reaction vessels, mixing vessels, and other components that facilitate the preparation of reagents or the test sample. The kit can also include one or more instruments for assisting with obtaining a test sample, such as a syringe, pipette, forceps, measured spoon, or the like.

### EXAMPLES

The following examples are provided for illustrative purposes only and are not intended to limit the scope of the invention.

### Example 1

### ELISA

The BC2 nanobody/BC2T platform was evaluated in the context of ELISA. ELISA typically requires (1) immobilization of a protein (“protein A”) onto a surface (2) incubation with a binding partner (“protein B”) equipped with a small peptide tag; (3) treatment with an antibody-reporter protein conjugate, which recognizes the peptide tag, and generates a signal following addition of a small-molecule substrate (FIG. 2(A)). HorseRadish Peroxidase (HRP) is commonly used as a reporter protein. A direct comparison between the BC2/BC2T platform and commercially available antibodies that bind the myc tag (SEQ ID NO. 2: EQKLISEEDL), or His6 (HHHHHH) was conducted.

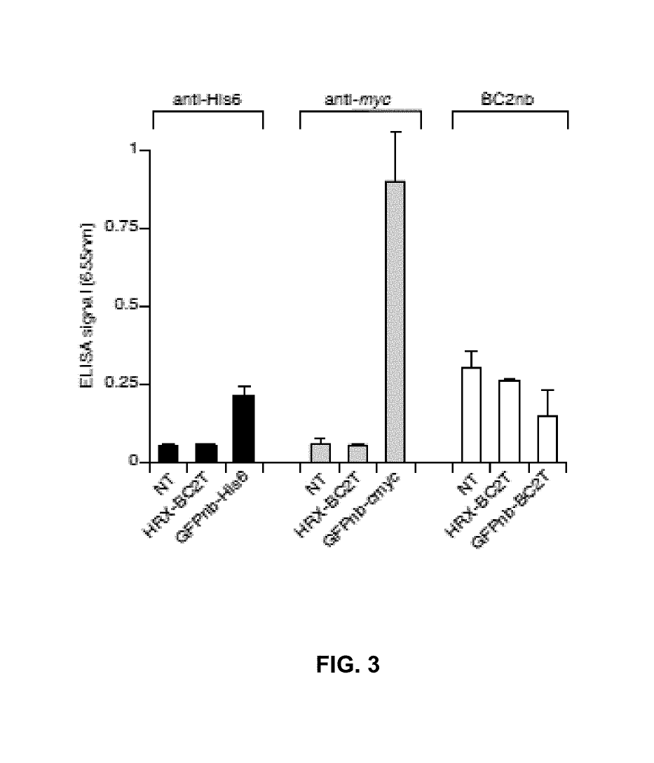

Green Fluorescent Protein (GFP) was immobilized onto the surface of a multi-well plate. Following a washing step, GFP-coated wells were treated with either buffer (NT), HRX-BC2T (which has no appreciable affinity for GFP), or a GFP-binding nanobody-His6 fusion protein (GFPnb-His6), which tightly binds GFP (K_{D}˜1 nM). After washing steps to remove unbound material, wells were incubated with a commercially available anti-His6 antibody-HRP conjugate, and HRP substrate. No appreciable signal is observed in wells incubated with HRX-BC2T (indicating no interaction between HRX and GFP, and no recognition of BC2T by anti-His6, FIG. 2(B), black). However, strong signal is generated in GFP immobilized wells following treatment with GFPnb-His6, and subsequent incubation with anti-His6-HRP and HRP substrate (FIG. 2(B), black). Similarly, when GFP immobilized wells are treated with buffer (NT), HRX-BC2T, or GFPnb-His6, no appreciable signal is observed after subsequent incubation with anti-myc-HRP and HRP substrate (FIG. 2(B), gray). However, signal that compares favorably to the analogous His6/anti-His6 experiment is observed in wells that contain immobilized GFP, following treatment with GFPnb-myc tag, and subsequent incubation with anti-myc-HRP and HRP substrate (FIG. 2(B), gray). Signal that compares favorably to the analogous experiments described above is observed when wells containing immobilized GFP are treated with GFPnb-BC2T, and subsequent incubation with ‘in house’ prepared BC2nb-HRP conjugate and HRP substrate (FIG. 2(B), white). As expected, no appreciable signal was observed when GFP containing wells were treated with buffer (NT), HRX-BC2T, or GFPnb-His6, following subsequent treatment with BC2nb-HRP and HRP substrate (FIG. 2(B), white). Additionally, no appreciable signal was observed in wells lacking immobilized GFP (FIG. 3).

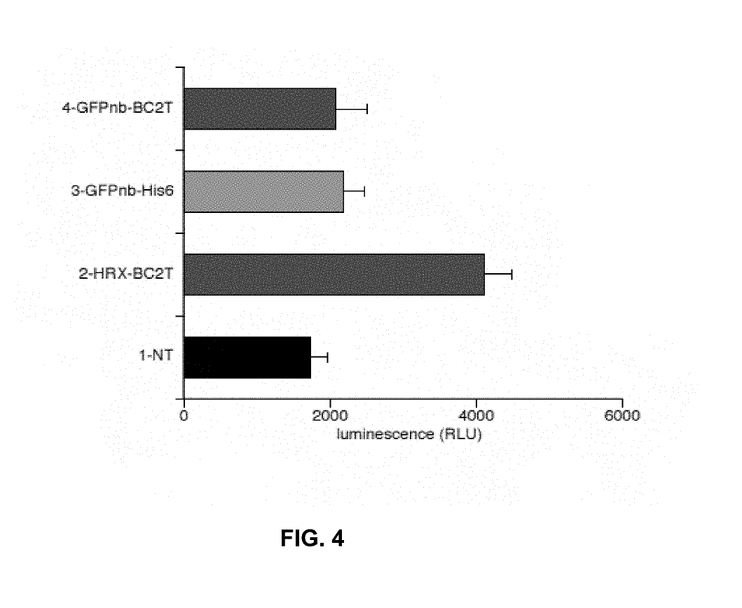

Chemical conjugation of HRP to BC2 may be an impediment to broad use of this reagent for laboratories without experience in bioconjugation. A more practical solution is expression of the BC2 nanobody as a fusion to a reporter protein. However, the BC2 nanobody-HRP fusion does not express as a soluble protein in E. coli. The bioluminescent ‘nanoluciferase’ protein nLuc (Promega) expresses as a fusion to BC2 nanobody. As such, a BC2 nanobody-nLuc fusion was created. The BC2 nanobody-nLuc performed well in the ELISA analysis. First, biotinylated GFP was immobilized onto streptavidin-coated plates. Wells containing immobilized GFP were then incubated with either buffer (NT), HRX-BC2T, GFPnb, or GBPnb-BC2T. Following washing steps to remove unbound material, wells were treated with the BC2-nLuc fusion protein, washed again, then treated with the nLuc substrate (“NanoGlo™”). No appreciable signal was generated in wells containing immobilized GFP, but incubated with either HRX or GFP-binding nanobody lacking the BC2T peptide (FIG. 2(C), HRX-BC2T and GFPnb, respectively). In contrast, robust signal was observed in lanes containing immobilized GFP in complex with the GFP-binding nanobody genetically fused to the BC2T peptide (FIG. 2(C), GFPnb-BC2T). When immobilized GFP was treated with a solution containing equal parts GFP-binding nanobody-BC2T peptide and GFPbinding nanobody (without the tag), a ˜50% decrease in luminescence is observed, compared to wells treated with only the GFPbinding nanobody equipped with the BC2T peptide (FIG. 2(C), 1:1 mixture). In contrast, no appreciable signal was observed in wells that were treated identically, but lack immobilized GFP (FIG. 4). Collectively, these data show that an ‘in house’ prepared BC2 nanobody-nLuc fusion protein, when paired with binding partners containing the BC2 tag, can be used for ELISA.

### Example 2

### Flow Cytometry

The BC2 nanobody/BC2T platform was evaluated in the context of flow cytometry—a commonly used technique to evaluate protein-protein and protein-nucleic acid interactions on the surface of yeast or bacteria, and enrichment of binders from a protein library by Fluorescence Activated Cell Sorting (FACS). In a typical flow cytometry experiment, bacteria or yeast display a peptide or protein that is flanked by a peptide tag recognized by a commercial antibody-fluorescent dye conjugate. Interaction between the tag and antibody-reporter conjugate allows researchers to quantitate display efficiency. Concomitantly, the peptide or protein displaying cells are treated with a binding target that is also fluorescently tagged.

Traditionally, yeast display efficiency has been measured using either a commercially available antibody-dye conjugate that binds to an N-terminal HA tag or a C-terminal myc tag. Bacterial display efficiency on E. coli is typically measured using a commercially antibody that binds to a C-terminal myc tag. To permit direct comparative analysis, bacteria (E. coli) were engineered to display a small (˜15 kDa) well behaved protein (monomeric streptavidin, mSA2), with flanking N-terminal and C-terminal BC2T and myc tags, respectively (FIG. 5(A)). Yeast were engineered to display a HA-mSA2-BC2T-myc fusion (FIG. 5(B)).

For E. coli, cells were induced to express the displayed protein/tag fusion (as a fusion to OmpX—an E. coli cell surface protein typically used for bacterial display), then treated with either a commercially available anti-myc-FITC antibody-fluorescent dye conjugate, ‘in house’ prepared BC2 nanobody-Cy5 conjugate (BC2nb-Cy5), or a BC2 nanobody-GFP fusion protein (BC2nb-GFP). Following washing steps to remove unbound material, cells were analyzed by flow cytometry, using a laser/detection channel specific to either Cy5 or FITC (GFP). Both the BC2nb-Cy5 conjugate and BC2nb-GFP fusion compared favorably to the anti-myc-FITC antibody-fluorescent dye conjugate (˜98% display efficiency for each , FIG. 5(C)). Co-treatment with equal parts anti-myc-FITC and BC2nb-Cy5 show essentially identical fluorescence (recognition of their respective displayed peptide tag (FIG. 5(C)). Representative flow cytometry histograms are provided FIG. 6(A) and FIG. 6(B).

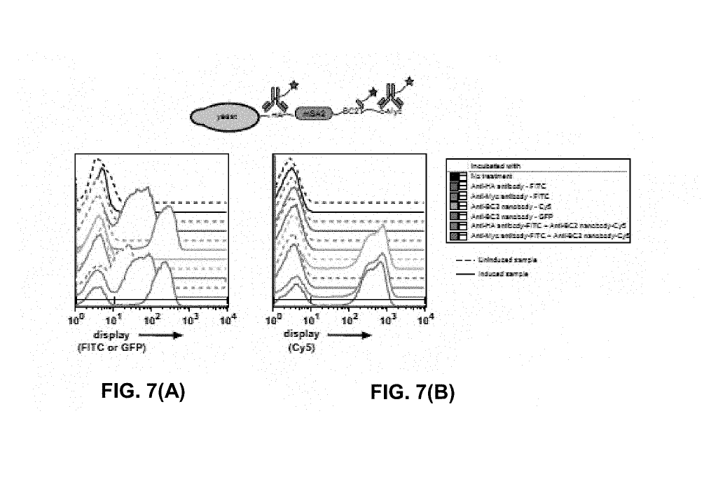

For yeast, cells were induced to express the displayed protein/tag fusion at the C-terminus of Aga2 (a yeast cell surface protein typically used for yeast display), then treated with either a commercially available anti-myc-FITC, anti-HA-FITC antibody-fluorescent dye conjugate, BC2nb-Cy5 conjugate, or BC2nb-GFP fusion. Again, the nanobody reagents compared favorably to commercially available antibody reagents. Individual treatment, or cotreatment with equal parts anti-myc-FITC and BC2nb-Cy5, or anti-HA-FITC and BC2nb-Cy5 show essentially identical fluorescence (recognition of their respective displayed peptide tag, FIG. 5(C)). Representative flow cytometry histograms are provided in FIG. 7(A) and FIG. 7(B).

### Example 3

### Western Blot

The utility of the BC2 nanobody/BC2T platform was assessed in Western blot—a commonly used technique to measure the presence of a specific protein (such as a tagged protein) in cell lysate. Execution of a Western blot typically requires: (1) denaturation of proteins from cell lysate; (2) separation of proteins based on their size via SDS-PolyAcrylamide Gel Electrophoresis (SDS-PAGE); (3) electrophoretic transfer of separated proteins to a membrane; (4) treatment of the protein-bound membrane with a primary antibody that either recognizes a specific protein, or a specific peptide tag, and; (5) treatment with a secondary antibody-dye conjugate, which serves to illuminate the primary antibody-bound protein. To function in this context, the BC2 nanobody must recognize the BC2T tag following a chemical denaturation step (and subsequent denaturation of the protein to which it is attached). For this reason, many antibodies are not suitable for Western blot analysis.

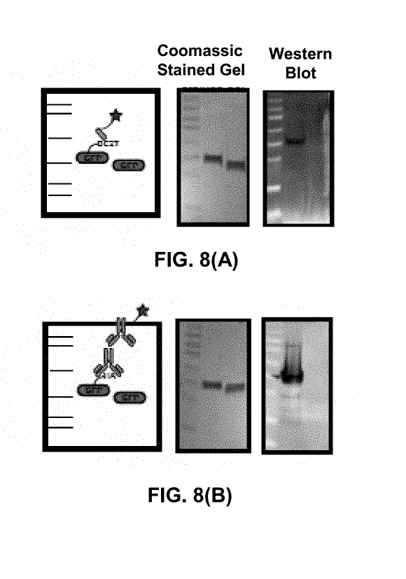

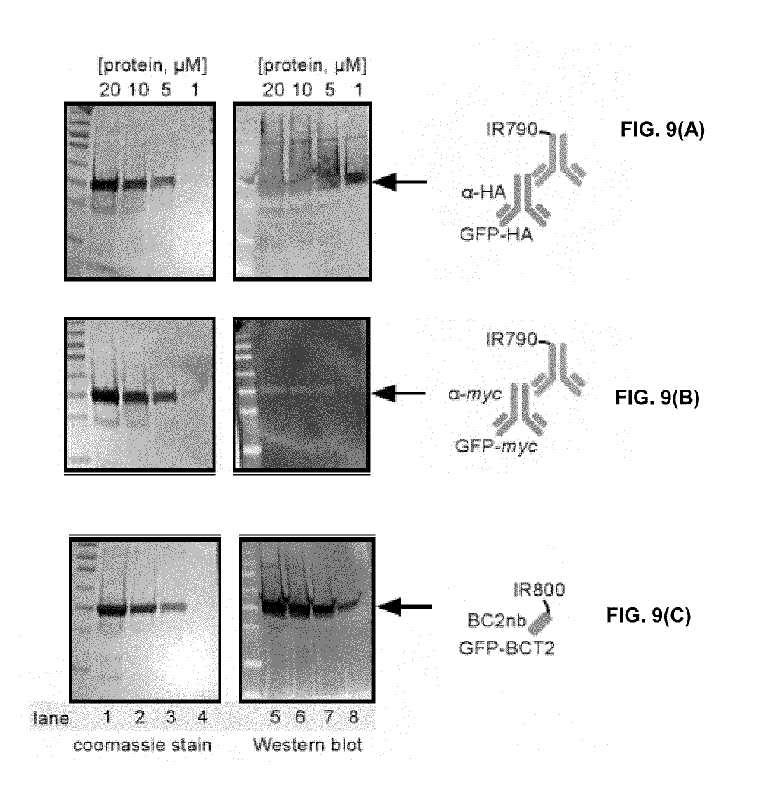

For comparison to IR dye 790-labelled commercially available secondary antibody, an IR dye 800-labelled BC2 nanobody conjugate was prepared by reaction between a C-terminal cysteine and commercially available dye-maleimide. First, 5 μM of GFP lacking the BC2T peptide, or GFP-BC2T was run on a polyacrylamide gel, transferred to PVDF membrane, and treated with BC2nb-IR800 reagent. Only GFP-BC2T was detected, but not GFP lacking BC2T peptide, indicating that recognition relies entirely on the nanobody-tag recognition, in this context (FIG. 8). Next, purified GFP-HA, GFP-myc, or GFP-BC2T were ran in duplicate on a polyacrylamide gel at 20 μM, 10 μM, 5 μM, and 1 μM concentrations. Following PAGE, one gel was stained by Coomassie to determine protein purity. Proteins embedded in the other gel were transferred onto a PVDF membrane. Membranes containing GFP-HA or GFP-myc were first treated with commercially available anti-HA or anti-myc primary antibodies suggested for Western blot experiments. Next, these membranes were treated with a secondary antibody-Alexa Fluor 790 dye. Following washing steps, membranes were imaged on a Li-Cor Odyssey instrument. All three proteins (GFP-HA, GFP-myc, or GFP-BC2T) were found to be pure, as determined by Coomassie staining (FIG. 9(A)-(C), left gels). As expected, both anti-HA and anti-myc antibodies recognize HA or myc tagged proteins in the Western blot (FIG. 9(A)-(B), right gels). The BC2 nanobody IR800 dye conjugate recognized GFP-BC2T with excellent potency and selectivity (FIG. 9(C), right gels). The BC2nb/BC2T pair generated a more robust and cleaner signal, in comparison to the HA and myc platforms.

### Methods

Cloning: Purified proteins. All plasmids were constructed on a pETDuet-1 backbone. All proteins were assembled from a set of overlapping oligonucleotides or purchased g-block. Constructs were amplified using vent and then ligated into NcoI and KpnI restriction enzyme cleavage sites in the pETDuet-1 plasmid.

Cloning: Display vectors. EBY100 yeast (trp-, leu-, with the Aga1p gene stably integrated) and pCTCON2 plasmid were generously provided by the Wittrup lab (MIT). The gene coding for mSA2 flagged with C-terminal BC2T were PCR amplified using vent and the constructs were ligated into NheI and BamHI restriction enzyme cleavage sites in the pCTCON2 plasmid. MC1061 bacteria electrocompetent cells and pB33-eCPX plasmid were generously provided by the Daugherty lab (UCSB). The gene coding for BC2T-mSA2-myc were PCR amplified using vent polymerase (NEB) and the constructs were ligated into NdeI and XhoI restriction enzyme cleavage sites in the pB33-eCPX plasmid.

Protein Purification. Plasmids were transformed into BL21s (DE3) (NEB). Cells were grown in either 2500 or 500 mL LB cultures containing carbenicillin (GoldBio Technology) at 37° C. to OD₆₀₀=˜0.5 and induced with 1 mM Isopropyl-β-D-1-thiogalactopyranoside (IPTG) (GoldBio Technology) at 20° C. overnight. Cells were then collected by centrifugation and resuspended in phosphate buffer with 2 M NaCl (20 mM Sodium Phosphate, pH 7.4) and stored at −20° C. Frozen pellets were thawed and incubated with cOmplete ULTRA protease inhibitors tablets, EDTA-free (Roche) then sonicated for 2 minutes. The lysate was cleared by centrifugation (8000 rpm, 20 minutes) and the supernatant was mixed with 1 mL Ni-NTA resin for 30 minutes. The resin was collected by centrifugation (4750 rpm, 10 minutes). The resin was washed with 50 mL buffer and 20 mM imidazole then 10 mL buffer and 50 mM imidazole. The protein was then eluted with 7 mL buffer containing 200 mM imidazole. The proteins were dialyzed against buffer with 150 mM NaCl and analyzed for purity by SDS-PAGE. Purified proteins were quantified using absorbance at 280 nm.

Protein Conjugation: Nanobody Dye Conjugation/HRP. Purified BC2 nanobodies with a C-terminal Cysteine residue were reacted with maleimide dye conjugates or maleimide HRP as described by manufacturer's instructions. Briefly, ˜10-20 fold molar excess of dye over protein was added to nanobody solution in PBS (Corning Cell Grow), mixed and incubated at room temperature for 2 hours to overnight. The dye labeled nanobody or HRP labeled nanobody was then purified by dialysis. It was stored, protected from light, at 4° C. until ready for use.

Protein Conjugation: Protein-Biotin conjugation. GFP was conjugated to biotin using Avidity BioMix protocols and purified BirA Protein Ligase (Avidity) at 1.0 mg/mL.

ELISA binding assay: HRP. ELISA assays were performed using clear, streptavidin coated, 96-well plates (Pierce). The plate was washed 3 times with wash buffer (20 mM phosphate, 150 mM NaCl, 0.05% Tween-20, and 0.1 mg/mL BSA, pH=7.4). Following washing, 100 μL of biotinylated GFP at 10 μg/mL was incubated for 2 hours at RT. Wells were washed three times with 200 μL of wash buffer shaking for 5 minutes. Subsequently, wells containing GFP were then incubated for 1 hour at RT with 100 μL of buffer containing one of three different proteins, all at 50 nM: (1) BC2 tagged protein that has no appreciable affinity for GFP (zinc finger protein HRX, referred to as HRX); (2) a GFP binding nanobody-His6 that tightly binds GFP (K_{D}˜1 nM), but lacks the BC2T epitope, or (3) GFP binding nanobody fused to a C-terminal BC2T or myc tag, then washed three times with 200 μL wash buffer. Following this, a 1:10,000 dilution of HRP-conjugated anti-His6× or anti-myc antibody were incubated in 100 μL Odyssey Blocking Buffer separately for all samples and ˜50nM solution of BC2nb-HRP in 100 μL Odyssey Blocking Buffer (LI-COR) for a separate set of all constructs for 1 hour at RT, and washed 3 times with 200 μL wash buffer. Colorimetry was developed for 30 minutes using 100 μL of TMB-One substrate (Promega). Absorbance was measured at 655 nm on a plate reader.

ELISA data using BC2 nanobody-HRP for reading is shown in FIG. 3. All lanes did not have anything immobilized on the plate's surface. To test non-specific binding of anti-His6 antibody-HRP, anti-myc antibody-HRP, and BC2nb-HRP, wells were then incubated with just buffer (NT), a protein that does not have affinity for GFP (HRX-BC2T), a protein that does have affinity for GFP but different tags depending on which antibody was used (anti-His6, colored black, anti-myc, colored grey, and BC2 nanobody-HRP, colored white). After a 30 minute incubation with TMB-one substrate plate was read at 655nm. All experiments were performed in triplicate. Error bars represent standard deviation of three experiments. NT=no treatment.

ELISA binding assay: NanoLuciferase. ELISA assays were performed using black, streptavidin coated, 96-well plates (Pierce). The plate was washed 3 times with wash buffer (20 mM phosphate, 150 mM NaCl, 0.05% Tween-20, and 0.1 mg/mL BSA, pH=7.4). Following washing, 100 μL of biotinylated GFP at 10 μg/mL was incubated for 2 hours at RT. Wells were washed 3 times with 200 μL wash buffer, shaking for 5 minutes. Subsequently, wells containing GFP were then incubated for 1 hour at RT with 100 μL of buffer containing one of three different proteins, all at 50 nM: (1) BC2 tagged protein that has no appreciable affinity for GFP (zinc finger protein HRX, referred to as HRX); (2) a GFP binding nanobody-His6 that tightly binds GFP (KD˜10 nM), but lacks the BC2T epitope, or (3) GFP binding nanobody fused to a C-terminal BC2T, then washed three times with 200 μL wash buffer. Following this, 50 nM of BC2-nanobody-nLuc fusion protein in wash buffer was incubated for 1 hour, and washed four times with 200 μL wash buffer. Finally, 100 μL of NanoGlo reagent substrate (Promega) diluted 1:50 in wash buffer was incubated with samples and allowed to shake for ˜10 min minutes at RT. Luminescence was measured on a plate reader.

ELISA data using BC2 nanobody-nLuc for reading is shown in FIG. 4. Lanes 1-4 did not have anything immobilized on the plate's surface. To test non-specific binding of BC2nb-nLuc, wells were then incubated with just buffer (NT; lane 1, colored black), a protein that does not have affinity for GFP (HRX-BC2T; lane 2, colored red), a protein that does have affinity for GFP but no BC2T epitope (GFPnb-His6; lane 3, colored orange), and GFP displaying BC2T (lane 4, colored green). Luminescence was read after a 10 minute incubation with NanoGlo substrate. All experiments were performed in triplicate. Error bars represent standard deviation of three experiments. NT=no treatment.

Flow Cytometry Analysis: Bacteria. 50 mL culture of bacteria displaying mSA2 with BC2T and myc were grown in a 250 mL baffled flask containing chloramphenicol (GoldBio Technologies) at 37° C. with shaking (250 rpm) until an OD₆₀₀=˜0.5 and induced with a final concentration of 0.02% (w/v) L-(+)-Arabinose (Sigma Aldrich) at 20° C. overnight with shaking (250 rpm). Approximately 10⁸ cells were pelleted and washed with 500 μL of 4° C. PBS-BSA. Bacteria were subsequently incubated with either BC2 nanobody—Cy5 (˜10 μg/mL), FITC-conjugated anti-myc antibody (1:10,000 dilution), or both BC2 nanobody-GFP alone in 500 μL PBS-BSA and rotated at RT for 1 hour. After incubation, two final washes with cold PBS-BSA were made to remove any unbound material and samples taken to flow cytometry analysis.

Table 1 shows Cy5, FITC, and GFP detected by flow cytometry to indicate display. All experiments were completed in triplicate. Values represent the mean of those experiments.

TABLE 1

| | | | | | FITC or |
|---|---|---|---|---|---|
| | Construct | Induced | Incubated with | Cy5 (+) | GFP (+) |
| 1 | Bacteria - | — | — | 0.87 | 1.73 |
| 2 | mSA2 | Yes | — | 1.72 | 1.23 |
| 3 | | — | Myc-ab-FITC | 1.17 | 0.82 |
| 4 | | Yes | Myc-ab-FITC | 3.11 | 94 |
| 5 | | — | BC2-nb Cy5 | 9.2 | 0.86 |
| 6 | | Yes | BC2-nb Cy5 | 98.6 | 1.9 |
| 7 | | — | BC2-nb-GFP | 1.09 | 11.5 |
| 8 | | Yes | BC2-nb-GFP | 3.68 | 98.1 |
| 9 | | Yes | BC2-nb Cy5 + | 98.7 | 93.1 |
| | | | Myc-ab-FITC | | |

Representative histogram of flow cytometry data for display of mSA2 on bacteria are shown in FIG. 6(A) & 6(B). In all cases, cases bacteria or yeast were detected after incubation with anti-HA antibody-FITC, anti-myc antibody-FITC, BC2 nanobody—Cy5, or BC2 nanobody-GFP. Non-induced samples are shown as dashed lines and induced samples as solid lines.

Flow Cytometry Analysis: Yeast. 50 mL culture of yeast displaying mSA2 with BC2T were grown in a 250 mL baffled flask containing SD-CAA for 2-3 days at 30° C. with shaking. After 2-3 days of growth in SD-CAA, the samples were subcultured in SD-CAA at an initial density of 1×10⁷ cells/mL and grown to a density of 2-5×10⁷ cells/mL. Yeast were subcultured again to a concentration of 1.0×10⁷ cells/mL in SG-CAA (Galactose containing induction media) and grown for 2 days shaking at 250 RPM at a temperature of 20° C. Approximately 10⁸ cells were pelleted and washed with 500 μL of 4° C. PBS-BSA. Yeast were subsequently incubated with either BC2 nanobody—Cy5 (˜10 μg/mL), FITC-conjugated anti-myc antibody/FITC conjugated anti-HA antibody (1:10,000 dilution), both the nanobody and one antibody, or BC2 nanobody—GFP (50 nM) alone in 500 μL PBS-BSA and rotated at room temperature for 1 hour. After incubation, two final washes with cold PBS-BSA were made to remove any unbound material and samples were taken to flow cytometry analysis.

Table 2 shows Cy5, FITC, and GFP detected by flow cytometry to indicate display. All experiments were performed in triplicate. Values represent the mean of those experiments.

TABLE 2

| | | | Incubated | | FITC or |
|---|---|---|---|---|---|
| | Construct | Induced | with | Cy5 (+) | GFP (+) |
| 1 | Yeast - | — | — | 0.63 | 6.11 |
| 2 | mSA2 | Yes | — | 0.17 | 1.1 |
| 3 | | — | Myc-ab-FITC | 0.27 | 0.67 |
| 4 | | Yes | Myc-ab-FITC | 0.53 | 71.7 |
| 5 | | — | HA-ab-FITC | 0.28 | 0.71 |
| 6 | | Yes | HA-ab-FITC | 0.58 | 69.3 |
| 7 | | — | BC2-nb Cy5 | 1.01 | 0.81 |
| 8 | | Yes | BC2-nb Cy5 | 71.1 | 1.61 |
| 9 | | — | BC2-nb-GFP | 0.28 | 0.82 |
| 10 | | Yes | BC2-nb-GFP | 0.57 | 59.6 |
| 11 | | Yes | BC2-nb Cy5 + | 70.9 | 71 |
| | | | Myc-ab-FITC | | |
| 12 | | Yes | BC2-nb Cy5 + | 71.2 | 69.8 |
| | | | HA-ab-FITC | | |

Representative histogram of flow cytometry data for display of mSA2 on yeast are shown in FIG. 7(A) & 7(A). In all cases, cases bacteria or yeast were detected after incubation with anti-HA antibody-FITC, anti-myc antibody-FITC, BC2 nanobody—Cy5, or BC2 nanobody-GFP. Non-induced samples are shown as dashed lines and induced samples as solid lines.

Western Blot Analysis: Using commercially available antibodies. Purified proteins were separated by SDS-PAGE and transferred to a PVDF membrane via an iBlot Western blot apparatus (Invitrogen). The membrane was blocked with 1×PBS, 5% milk and 0.1% Tween-20 for 1 hour at RT. Primary antibodies for myc and HA tag were incubated separately with the appropriate membranes overnight at a 1:10,000 dilution in 10 mL of 1×PBS, 5% BSA, and 0.1% Tween-20 at 4° C. Membranes were washed 3× with 1×PBS containing 0.1% Tween-20 and then incubated with Anti-Rabbit (Alexa Fluor 790) at a 1:10,000 dilution in 10 mL PBS, 5% milk and 0.1% Tween-20 for 1 hour at RT. The membranes were then washed 3× with 1×PBS containing 0.1% Tween-20 and imaged in 1×PBS using the Odyssey Classic Infrared Imager (LI-COR).

Western Blot Analysis: Using ‘in-house’ prepared nanobody-IR800 dye. Purified proteins were separated by SDS-PAGE and transferred to a PVDF membrane via an iBlot Western blot apparatus. The membrane was incubated with 1×PBS, 5% milk, and 0.1% Tween-20 for 1 hour at RT. The BC2 nanobody-IR800 dye conjugate was then incubated overnight at ˜0.10 μM concentration in 10 mL of 1×PBS, 5% BSA, and 0.1% Tween-20 at 4° C. The membrane was then washed 3× with 1×PBS containing 0.1% Tween-20 and imaged in 1×PBS using the Odyssey Classic Infrared Imager. FIG. 8(A) shows selectivity for the epitope of GFP-BC2T. Western blot analysis used BC2 nanobody-IRdye800. FIG. 8(B) shows selectivity for the epitope of GFP-HA. Western blot analysis used anti-HA antibody and was visualized with Donkey Anti-Rabbit IgG Alexa Fluor 790.

All publications and patent applications cited in this specification are herein incorporated by reference as if each individual publication or patent application were specifically, and individually, indicated to be incorporated by reference.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed as the best mode contemplated for carrying out this invention, but that the invention will include all embodiments falling within the scope of the appended claims.

### REFERENCES

Byrne, B.; Stack, E.; Gilmartin, N.; O'Kennedy, R., Antibody-based sensors: principles, problems and potential for detection of pathogens and associated toxins. Sensors (Basel) 2009, 9 (6), 4407-45.

Holford, T. R.; Davis, F.; Higson, S. P., Recent trends in antibody based sensors. Biosens Bioelectron 2012, 34 (1), 12-24.

Liu, Z. Q.; Mahmood, T.; Yang, P. C., Western blot: technique, theory and trouble shooting. N Am J Med Sci 2014, 6 (3), 160.

The enzyme-linked immunosorbent assay (ELISA). Bull World Health Organ 1976, 54 (2), 129-39.

Laerum, O. D.; Farsund, T., Clinical application of flow cytometry: a review. Cytometry 1981, 2 (1), 1-13.

Carlsson, H. E.; Hurvell, B.; Lindberg, A. A., Enzyme-linked immunosorbent assay (ELISA) for titration of antibodies against Brucella abortus and Yersinia enterocolitica. Acta Pathol Microbiol Scand C 1976, 84 (3), 168-76.

Einhauer, A.; Jungbauer, A., The FLAG peptide, a versatile fusion tag for the purification of recombinant proteins. J Biochem Biophys Methods 2001, 49 (1-3), 455-65.

Evan, G. I.; Lewis, G. K.; Ramsay, G.; Bishop, J. M., Isolation of monoclonal antibodies specific for human c-myc proto-oncogene product. Mol Cell Biol 1985, 5 (12), 3610-6.

Schmidt, T.; Skerra, A., The Strep-tag system for one-step affinity purification of proteins from mammalian cell culture. Methods Mol Biol 2015, 1286, 83-95.

Wilson, I. A.; Niman, H. L.; Houghten, R. A.; Cherenson, A. R.; Connolly, M. L.; Lerner, R. A., The structure of an antigenic determinant in a protein. Cell 1984, 37 (3), 767-78.

Bruce, V. J.; Ta, A. N.; McNaughton, B. R., Minimalist Antibodies and Mimetics: An Update and Recent Applications. Chembiochem 2016, 17 (20), 1892-1899.

Muyldermans, S., Nanobodies: natural single-domain antibodies. Annu Rev Biochem 2013, 82, 775-97.

Gray, M. A.; Tao, R. N.; DePorter, S. M.; Spiegel, D. A.; McNaughton, B. R., A Nanobody Activation Immunotherapeutic that Selectively Destroys HER2-Positive Breast Cancer Cells. Chembiochem 2016, 17 (2), 155-8.

Schoonooghe, S.; Laoui, D.; Van Ginderachter, J. A.; Devoogdt, N.; Lahoutte, T.; De Baetselier, P.; Raes, G., Novel applications of nanobodies for in vivo bio-imaging of inflamed tissues in inflammatory diseases and cancer. Immunobiology 2012, 217 (12), 1266-72.

Buchfellner, A.; Yurlova, L.; Nuske, S.; Scholz, A. M.; Bogner, J.; Ruf, B.; Zolghadr, K.; Drexler, S. E.; Drexler, G. A.; Girst, S.; Greubel, C.; Reindl, J.; Siebenwirth, C.; Romer, T.; Friedl, A. A.; Rothbauer, U., A New Nanobody-Based Biosensor to Study Endogenous PARP1 In Vitro and in Live Human Cells. PLoS One 2016, 11 (3), e0151041.

Braun, M. B.; Traenkle, B.; Koch, P. A.; Emele, F.; Weiss, F.; Poetz, O.; Stehle, T.; Rothbauer, U., Peptides in headlock—a novel high-affinity and versatile peptide-binding nanobody for proteomics and microscopy. Sci Rep 2016, 6, 19211.

Kubala, M. H.; Kovtun, O.; Alexandrov, K.; Collins, B. M., Structural and thermodynamic analysis of the GFP:GFP-nanobody complex. Protein Sci 2010, 19 (12), 2389-401.

Hall, M. P.; Unch, J.; Binkowski, B. F.; Valley, M. P.; Butler, B. L.; Wood, M. G.; Otto, P.; Zimmerman, K.; Vidugiris, G.; Machleidt, T.; Robers, M. B.; Benink, H. A.; Eggers, C. T.; Slater, M. R.; Meisenheimer, P. L.; Klaubert, D. H.; Fan, F.; Encell, L. P.; Wood, K. V., Engineered luciferase reporter from a deep sea shrimp utilizing a novel imidazopyrazinone substrate. ACS Chem Biol 2012, 7 (11), 1848-57.

Lofblom, J., Bacterial display in combinatorial protein engineering. Biotechnol J 2011, 6 (9), 1115-29.

Daugherty, P. S., Protein engineering with bacterial display. Curr Opin Struct Biol 2007, 17 (4), 474-80.

Laplagne, D. A.; Zylberman, V.; Ainciart, N.; Steward, M. W.; Sciutto, E.; Fossati, C. A.; Goldbaum, F. A., Engineering of a polymeric bacterial protein as a scaffold for the multiple display of peptides. Proteins 2004, 57 (4), 820-8.

Boder, E. T.; Wittrup, K. D., Yeast surface display for directed evolution of protein expression, affinity, and stability. Methods Enzymol 2000, 328, 430-44.

Kieke, M. C.; Shusta, E. V.; Boder, E. T.; Teyton, L.; Wittrup, K. D.; Kranz, D. M., Selection of functional T cell receptor mutants from a yeast surface-display library. Proc Natl Acad Sci USA 1999, 96 (10), 5651-6.

Boder, E. T.; Wittrup, K. D., Yeast surface display for screening combinatorial polypeptide libraries. Nat Biotechnol 1997, 15 (6), 553-7.

What is claimed is:

1. A method of detecting a tagged target comprising:
obtaining a nanobody having binding affinity for a tag, wherein the nanobody is linked to a reporter;
contacting the tagged target with the nanobody, wherein the tag is present on the target; and
detecting the reporter.

2. The method according to claim 1, wherein the target is selected from a protein, protein fragment, peptide, amino acid, and cell.

3. The method according to claim 1, wherein the tag is a peptide having 6 to 20 amino acids.

4. The method according to claim 3, the peptide having at least 80% identity to the amino acid sequence SEQ ID NO. 1: PDRKAAVSHWQQ.

5. The method according to claim 4, the peptide having the amino acid sequence SEQ ID NO. 1: PDRKAAVSHWQQ.

6. The method according to claim 1, wherein the reporter is selected from a reporter protein, dye, and radioisotope.

7. The method according to claim 6, wherein the reporter protein is selected from fluorescent protein, luciferase, alkaline phosphatase, β-galactosidase, β-lactamase, dihydrofolate reductase, and ubiquitin.

8. The method according to claim 7, wherein the reporter protein is a luciferase.

9. The method according to claim 8, wherein the luciferase is nLuc.

10. A method of detecting a cell comprising:
obtaining a cell that has been modified to display a tagged protein;
contacting the tagged protein with a nanobody linked to a reporter, wherein the nanobody binds the tag on the tagged protein; and
detecting the reporter.

11. The method according to claim 10, wherein the tag is a peptide having 6 to 20 amino acids.

12. The method according to claim 11, the peptide having at least 80% identity to the amino acid sequence SEQ ID NO. 1: PDRKAAVSHWQQ.

13. The method according to claim 12, the peptide having the amino acid sequence SEQ ID NO. 1: PDRKAAVSHWQQ.

14. The method according to claim 10, wherein the reporter selected from a reporter protein, dye, and radioisotope.

15. The method according to claim 14, wherein the reporter protein is selected from fluorescent protein, luciferase, alkaline phosphatase, β-galactosidase, β-lactamase, dihydrofolate reductase, and ubiquitin.

16. The method according to claim 15, wherein the reporter protein is a luciferase.

17. The method according to claim 16, wherein the luciferase is nLuc.

18. A nanobody-peptide tag receptor system comprising a tagged target and a nanobody linked to a reporter, wherein the nanobody has binding affinity to the tag on the tagged target.

19. The nanobody-peptide tag receptor system according to claim 18, wherein the target is selected from a protein, protein fragment, peptide, amino acid, and cell.

20. The nanobody-peptide tag receptor system according to claim 18, wherein the tag is a peptide having 6 to 20 amino acids.

21. The nanobody-peptide tag receptor system according to claim 20, the peptide having at least 80% identity to the amino acid sequence SEQ ID NO. 1: PDRKAAVSHWQQ.

22. The nanobody-peptide tag receptor system according to claim 21, the peptide having the amino acid sequence SEQ ID NO. 1: PDRKAAVSHWQQ.

23. The nanobody-peptide tag receptor system according to claim 18, wherein the reporter is selected from a reporter protein, dye, and radioisotope.

24. The nanobody-peptide tag receptor system according to claim 23, wherein the reporter protein is selected from fluorescent protein, luciferase, alkaline phosphatase, β-galactosidase, β-lactamase, dihydrofolate reductase, and ubiquitin.

25. The nanobody-peptide tag receptor system according to claim 24, wherein the reporter protein is a luciferase.

26. The nanobody-peptide tag receptor system according to claim 25, wherein the luciferase is nLuc.

27. The nanobody-peptide tag receptor system according to claim 18, wherein the system is used in an immunological method selected from immunoassays, indirect immunofluorescence, direct immunofluorescence, enzyme-linked immunosorbent assay (ELISA), flow cytometry, fluorescence activated cell sorting (FACS), Western blot, paper-based diagnostics, and microfluidic diagnostics.
